(19) United States
(12) Patent Application Publication
(10) Pub. No.: US 2018/0140707 A1
(43) Pub. Date: May 24, 2018

(54) THERAPEUTIC MIXTURE COMPRISING AN HERBAL COMPOUND

(71) Applicant: Adam C Saucedo, Sandy, UT (US)

(72) Inventor: Adam C Saucedo, Sandy, UT (US)

(21) Appl. No.: 15/822,094

(22) Filed: Nov. 24, 2017

Related U.S. Application Data
(60) Provisional application No. 62/426,141, filed on Nov. 23, 2016.

(51) Int. Cl. A61K 47/40; B01J 19/24; B01J 19/00

(52) U.S. Cl. CPC A61K 47/40; B01J 19/24; B01J 2219/00186; B01J 2219/24; B01J 19/0006

(57) ABSTRACT

Disclosed is a method of preparing a therapeutic mixture including an herbal compound. Further, the method may include selecting, using a first selector unit, a cyclodextrin compound. Further, the method may include selecting, using a second selector unit, a therapeutic additive including the herbal compound. Yet further, the method may include determining, using a control unit, each of a first quantity of cyclodextrin compound and a second quantity of the therapeutic additive corresponding to a predetermined level of complexing of cyclodextrin compound. Moreover, the method may include complexing, using a mixer unit, the first quantity of cyclodextrin compound and the second quantity of the therapeutic additive to form the therapeutic mixture.

The current application claims a priority to the U.S. Provisional Patent application Ser. No. 62/426,141 filed on Nov. 23, 2016.

### FIELD OF THE INVENTION

The present invention relates generally to a therapeutic mixture. In particular, the present invention relates to an apparatus and a method for making a therapeutic mixture that protects and enhances bioavailability of herbal derivative substances.

### BACKGROUND OF THE INVENTION

Humans may intake various types of medications or nutrients to enhance their physical and mental well-being. However, many compounds in the medications or nutrients are filtered out by the kidneys before they can have any effect on the human body. Often, such compounds are herbal derivatives. Such compounds are found to have a low efficacy.

Accordingly, there is a need for improved methods and apparatus for increasing bioavailability of medications or nutrients that may also overcome one or more of the abovementioned problems and/or limitations.

### SUMMARY OF THE INVENTION

This summary is provided to introduce a selection of concepts in a simplified form that are further described below in the Detailed Description. This summary is not intended to identify key features or essential features of the claimed subject matter. Nor is this summary intended to be used to limit the claimed subject matter's scope.

Disclosed is a method of preparing a therapeutic mixture including an herbal compound. Further, the method may include selecting, using a first selector unit, a cyclodextrin compound. Further, the method may include selecting, using a second selector unit, a therapeutic additive including the herbal compound. Yet further, the method may include determining, using a control unit, each of a first quantity of cyclodextrin compound and a second quantity of the therapeutic additive corresponding to a predetermined level of complexing of the cyclodextrin compound. Moreover, the method may include complexing, using a mixer unit, the first quantity of cyclodextrin compound and the second quantity of the therapeutic additive to form the therapeutic mixture.

According to further aspects, an apparatus for preparing a therapeutic mixture including an herbal compound is disclosed. Further, the apparatus may include a first selector unit configured for selecting a cyclodextrin compound. Further, the apparatus may include a second selector unit configured for selecting a therapeutic additive including the herbal compound. Yet further, the apparatus may include a control unit configured for determining each of a first quantity of cyclodextrin compound and a second quantity of the therapeutic additive corresponding to a predetermined level of complexing of the cyclodextrin compound. Moreover, the apparatus may include a mixer unit configured for complexing the first quantity of cyclodextrin compound and the second quantity of the therapeutic additive to form the therapeutic mixture.

Additionally, in some embodiments, the mixer unit may be configured for complexing a third quantity of cyclodextrin compound with at least one of an omega fatty acid, an essential oil and a fat soluble vitamin.

According to some aspects, a therapeutic mixture including an herbal compound is disclosed. Further, the therapeutic mixture may include a first quantity of cyclodextrin compound and a second quantity of a therapeutic additive including the herbal compound. Further, each of the first quantity and the second quantity may correspond to a predetermined level of complexing of cyclodextrin compound with the therapeutic additive.

According to some aspects, a method of treating a disease is provided. The method may include administering to a subject in need thereof an effective amount of a therapeutic mixture including a first quantity of cyclodextrin compound and a second quantity of a therapeutic additive including an herbal compound. Further, each of the first quantity and the second quantity may correspond to a predetermined level of complexing of cyclodextrin compound with the therapeutic additive. Further, the disease may be one or more of obesity, type II diabetes, and dyslipidaemia.

According to some aspects, the present disclosure relates to a nutraceutical formulation including a nutrient and a therapeutic mixture including a first quantity of cyclodextrin compound and a second quantity of a therapeutic additive including an herbal compound. Further, each of the first quantity and the second quantity may correspond to a predetermined level of complexing of cyclodextrin compound with the therapeutic additive.

According to some embodiments, the present disclosure relates to a method for making a therapeutic mixture that protects and enhances the bioavailability of an herb or herbal derivative substance(s) through complexing with cyclodextrin, as well as enhancing the nutrient bioavailability properties of the herb or herbal derivatives and substances themselves. The therapeutic mixture of the cyclodextrin and herb or herbal derivative may also convey multiple additional properties related to protecting the herb or herbal derivative substance complexed with the cyclodextrin during transport and storage of the compound related to environmental variations. The cyclodextrin-complexed therapeutic compound may also include certain medications and nutrient substances which due to their hydrophobic properties, in the same manner as the herb or herbal derivatives, would couple with cyclodextrin rendering similar properties to the obtained therapeutic complex as described for the herb or herbal derivative substance(s).

Further, by complexing the herbal or herbal derived nutrient bio-enhancing substances with cyclodextrin and adding the therapeutic complex to food meals or to nutritional supplements (or other nutrient products) it enhances the bioavailability of the accompanying nutrients, once the cyclodextrin has released the inclusion compound such as herb or herbal derivative substance(s) in the body, and in particular, in the small intestine. It also provides many other advantages which protect the herb or herbal derivative from both environmental threats such as heat-related decomposition, microbiological contamination, and protection, once the product containing the cyclodextrin-complexed compound is taken by a consumer. It protects the herb or herbal derivative from the harsh environment of the stomach and intestine as the complexed product is delivered to the small intestine, where the inclusion substance such as herb or herbal derivative is release or uncoupled due to degradation of the cyclodextrin.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a block diagram of an apparatus for preparing a therapeutic mixture comprising an herbal compound, in accordance with some embodiments.

FIG. 2 illustrates a flowchart of a method for preparing a therapeutic mixture comprising an herbal compound, in accordance with some embodiments.

FIG. 3 illustrates a flowchart of a method for preparing a therapeutic mixture comprising an herbal compound, in accordance with some embodiments.

FIG. 4 illustrates a flowchart of a method for preparing a therapeutic mixture comprising an herbal compound, in accordance with some embodiments.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of enhancing bioavailability of certain compounds, embodiments of the present disclosure are not limited to use only in this context.

### Overview

According to some aspects, the present disclosure provides a cyclodextrin-complexed therapeutic compound. Further, the present disclosure provides a method for making a substance that increases the bioavailability of nutrients through the use of specific cyclodextrin-complexed herbs or herbal derivatives as well as provides protection of the inclusion substance from varying environmental parameters during transport and storage. First, the desired cyclodextrin variant may be selected. The user may then select one therapeutic additive (specifically an herb, herbal derivative or described substance). Next, the user may determine the amount of cyclodextrin variant and therapeutic additive required for the mixture to achieve maximal complexing of cyclodextrin and the inclusion substance such as herb, herbal derivative or other hydrophobic substance. Finally, the user may combine the desired quantity of therapeutic additive and cyclodextrin into a complexation of both. The disclosed method yields a therapeutic complex that is designed to be more effective than each of the individual compounds.

Further, the user may select a desired cyclodextrin compound from the variety of available cyclodextrin variants. These variants may include, but are not limited to, cyclodextrin alpha, cyclodextrin beta, and cyclodextrin gamma. The choice of cyclodextrin variant enables the user to select the composition most appropriate for the desired usage. Moreover, a therapeutic additive is a compound, which may be added by the user to a chosen cyclodextrin variant. The therapeutic compound may be an herb or herbal derivative compound or appropriate medication.

According to some aspects, the disclosure provides a method for making a therapeutic compound that includes one or more compounds known to have health benefits through nutrient bioavailability enhancement and to protect the herb or herbal derivative from the harsh environment of the stomach and intestine to deliver more intact and non-degraded inclusion substance in order to improve delivery of nutrients for enhanced bioavailability. Accordingly, the cyclodextrin and the therapeutic additive may be mixed into a carrying compound. The carrying compound may be a substance that enables the cyclodextrin and herbal or herbal derivative complexed compound to be easily administered to a user. The carrying compound enables delivery of the cyclodextrin-complexed therapeutic compound in one or more of a pill form, an injection, a syrup, a food item, or a product.

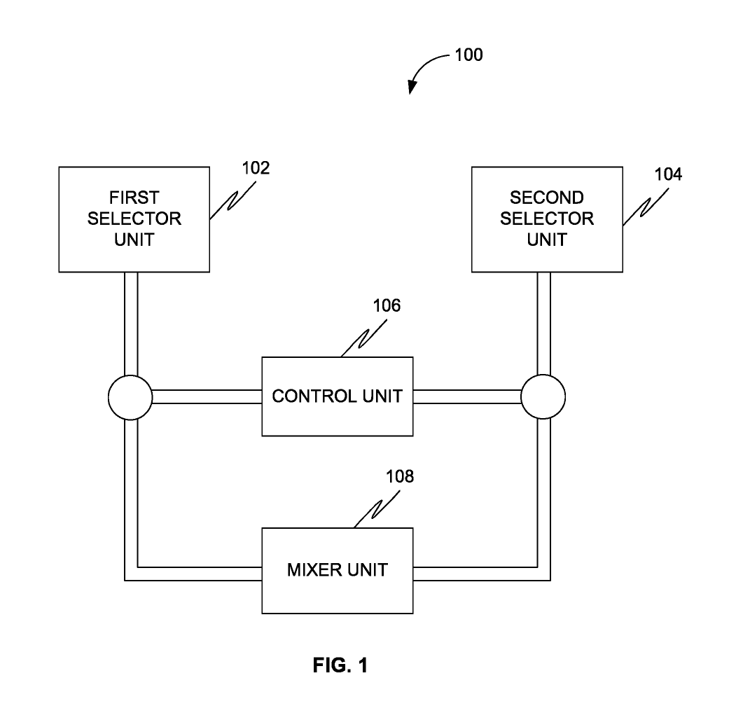

Referring now to figures, FIG. 1 is a block diagram of an apparatus 100 for preparing a therapeutic mixture comprising an herbal compound, in accordance with some embodiments. The herbal compound may include one or more of an herb and an herbal derivative. For example, the herbal compound may include one or more of Piperine, Reseveratrol, Coenzyme Q10, Niaziridin, Glycyrrhizin, Cuminum Cyminum, Carum Carvi, Alicillin, Alicillin & Cu2+, Lysergol, Aloe Vera Gel, Aloe Vera whole leaf extract, Stevia Rebaudiana, Curcumin, Sinomenine, Genistein, Ammannia Multiflora, Capsaicin, Quercetin and Naringin.

The apparatus 100 may include a first selector unit 102 configured for selecting a cyclodextrin compound. For example, the cyclodextrin compound may include one or more of cyclodextrin alpha, cyclodextrin beta, and cyclodextrin gamma.

In some embodiments, the cyclodextrin compound may include alpha cyclodextrin. Further, the first quantity of cyclodextrin compound may be determined to effect one or more of lowering a glycemic index of a food, lowering blood glucose level, lowering of Low-density lipoprotein (LDL), lowering of triglycerides and a weight loss in a user consuming the therapeutic mixture.

Further, the apparatus 100 may include a second selector unit 104 configured for selecting a therapeutic additive including the herbal compound.

Yet further, the apparatus 100 may include a control unit 106 configured for determining each of a first quantity of cyclodextrin compound and a second quantity of the therapeutic additive corresponding to a predetermined level of complexing of cyclodextrin compound. Further, the predetermined level of complexing of the cyclodextrin compound may include a maximum level.

Moreover, the apparatus 100 may include a mixer unit 108 configured for complexing the first quantity of the cyclodextrin compound and the second quantity of the therapeutic additive to form the therapeutic mixture.

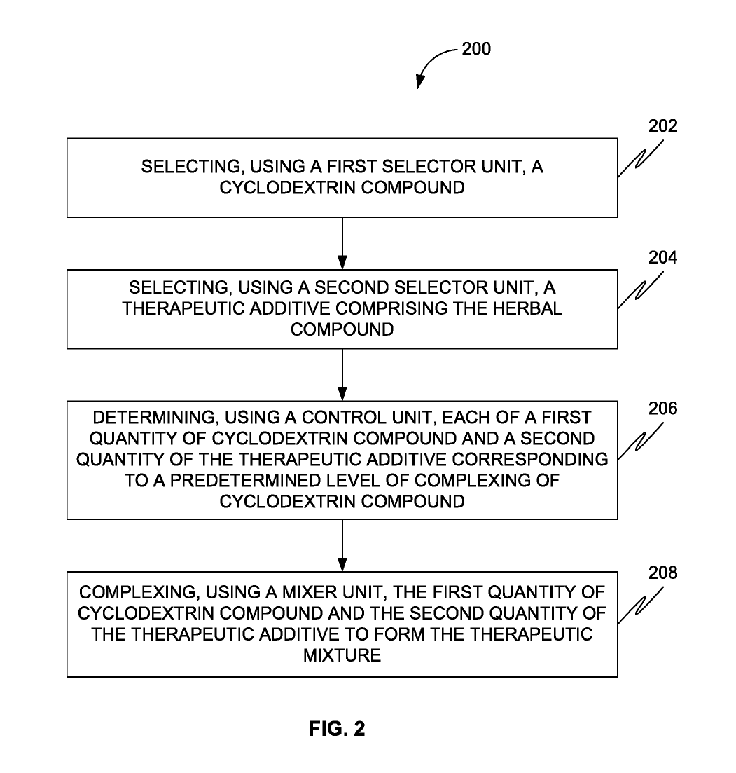

FIG. 2 illustrates a flowchart of a method 200 for preparing a therapeutic mixture comprising an herbal compound, in accordance with some embodiments. At 202, the method 200 may include selecting, using a first selector unit (such as the first selector unit 102), a cyclodextrin compound. Then, at 204, the method 200 may include selecting, using a second selector unit (such as the second selector unit 104), a therapeutic additive including the herbal compound.

Further, at 206, the method 200 may include may include determining, using a control unit (such as the control unit 106), each of a first quantity of cyclodextrin compound and a second quantity of the therapeutic additive corresponding to a predetermined level of complexing of cyclodextrin compound.

In some embodiments, the cyclodextrin compound may include alpha cyclodextrin. Further, the first quantity of cyclodextrin compound may be determined to effect one or more of lowering a glycemic index of a food, lowering blood glucose level, lowering of Low-density lipoprotein (LDL), lowering of triglycerides and a weight loss in a user consuming the therapeutic mixture. In further embodiments, the predetermined level of complexing of cyclodextrin compound may include a maximum level.

At 208, the method 200 may include complexing, using a mixer unit (such as the mixer unit 108), the first quantity of cyclodextrin compound and the second quantity of the therapeutic additive to form the therapeutic mixture.

In further embodiments, one or more of the selecting of the cyclodextrin compound (at 202) and determining the first quantity of cyclodextrin compound (at 206) may be performed to provide protection of the therapeutic mixture from one or more environmental disturbances. For example, the one or more environmental disturbances may include an oxidation, a light-induced reaction, a heat promoted destabilization, a loss by volatility, a sublimation, a microbiological contamination, a hygroscopicity, a masking of taste and a masking of flavor.

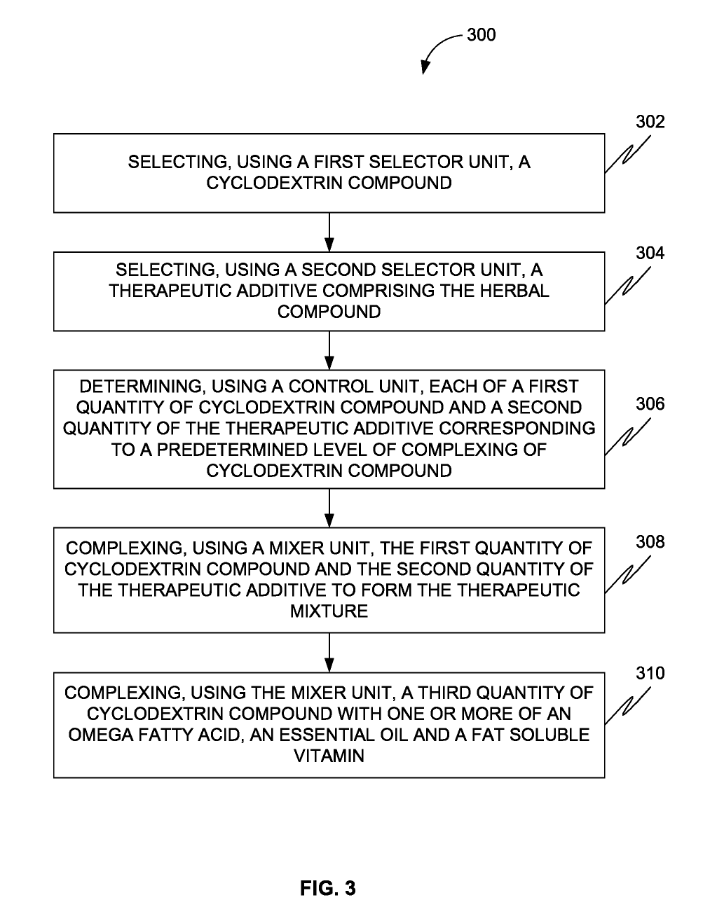

FIG. 3 illustrates a flowchart of a method 300 for preparing a therapeutic mixture comprising an herbal compound, in accordance with some embodiments. At 302, the method 300 may include selecting, using a first selector unit (such as the first selector unit 102), a cyclodextrin compound. Then, at 304, the method 300 may include selecting, using a second selector unit (such as the second selector unit 104), a therapeutic additive including the herbal compound. Further, at 306, the method 300 may include may include determining, using a control unit (such as the control unit 106), each of a first quantity of cyclodextrin compound and a second quantity of the therapeutic additive corresponding to a predetermined level of complexing of cyclodextrin compound. Then, at 308, the method 300 may include complexing, using a mixer unit (such as the mixer unit 108), the first quantity of cyclodextrin compound and the second quantity of the therapeutic additive to form the therapeutic mixture.

Yet further, at 310, the method 300 may include complexing using the mixer unit, a third quantity of cyclodextrin compound with one or more of an omega fatty acid, an essential oil, and a fat-soluble vitamin.

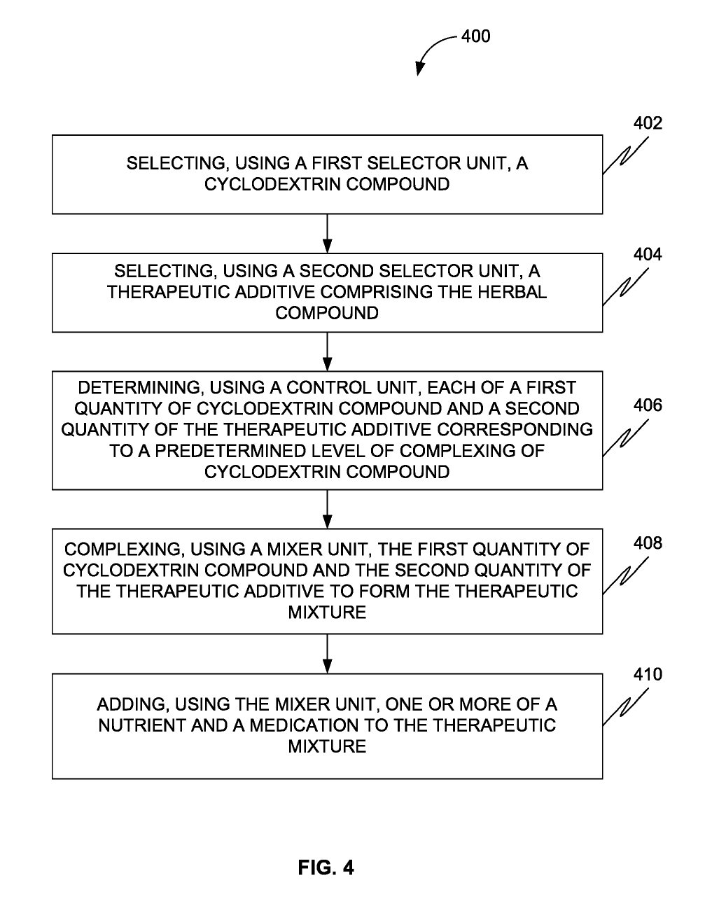

FIG. 4 illustrates a flowchart of a method 400 for preparing a therapeutic mixture comprising an herbal compound, in accordance with some embodiments. At 402, the method 400 may include selecting, using a first selector unit (such as the first selector unit 102), a cyclodextrin compound. Then, at 404, the method 400 may include selecting, using a second selector unit (such as the second selector unit 104), a therapeutic additive including the herbal compound. Further, at 406, the method 400 may include may include determining, using a control unit (such as the control unit 106), each of a first quantity of cyclodextrin compound and a second quantity of the therapeutic additive corresponding to a predetermined level of complexing of the cyclodextrin compound. Then, at 408, the method 400 may include complexing, using a mixer unit (such as the mixer unit 108), the first quantity of cyclodextrin compound and the second quantity of the therapeutic additive to form the therapeutic mixture.

Yet further, at 410, the method 400 may include adding, using the mixer unit, one or more of a nutrient and a medication to the therapeutic mixture. Further, the herbal compound enhances bioavailability of one or more of the nutrient and the medication. The method 400 may further include complexing, using the mixer unit, a fourth quantity of cyclodextrin compound with one or more of the nutrient and the medication.

In some embodiments, a therapeutic mixture including an herbal compound is disclosed. The herbal compound may include one or more of an herb and an herbal derivative. For example, the herbal compound may include one or more of Piperine, Reseveratrol, Coenzyme Q10, Niaziridin, Glycyrrhizin, Cuminum Cyminum, Carum Carvi, Alicillin, Alicillin & Cu2+, Lysergol, Aloe Vera Gel, Aloe Vera whole leaf extract, Stevia Rebaudiana, Curcumin, Sinomenine, Genistein, Ammannia Multiflora, Capsaicin, Quercetin and Naringin.

Further, the therapeutic mixture may include a first quantity of cyclodextrin compound and a second quantity of a therapeutic additive including the herbal compound. For example, the cyclodextrin compound may include one or more of cyclodextrin alpha, cyclodextrin beta, and cyclodextrin gamma.

Further, each of the first quantity and the second quantity may correspond to a predetermined level of complexing of cyclodextrin compound with the therapeutic additive. In further embodiments, the predetermined level of complexing of cyclodextrin compound may include a maximum level.

In further embodiments, the therapeutic mixture may include one or more of a nutrient and a medication. Further, the herbal compound may enhance bioavailability of one or more of the nutrient and the medication.

In further embodiments, the therapeutic mixture may include a fourth quantity of cyclodextrin compound complexed with one or more of the nutrient and the medication.

In further embodiments, the cyclodextrin compound may include alpha cyclodextrin. Further, the first quantity of cyclodextrin compound may be adequate to effect one or more of lowering a glycemic index of a food, lowering blood glucose level, lowering of Low-density lipoprotein (LDL), lowering of triglycerides and a weight loss in a user consuming the therapeutic mixture.

In further embodiments, the therapeutic mixture may include a third quantity of cyclodextrin compound complexed with one or more of an omega fatty acid, an essential oil, and a fat-soluble vitamin.

In further embodiments, the first quantity of cyclodextrin compound may be adequate to provide a predetermined level of protection of the therapeutic mixture from one or more environmental disturbances.

According to some embodiments, a method of treating a disease is provided. The method may include administering to a subject in need thereof an effective amount of a therapeutic mixture including a first quantity of cyclodextrin compound and a second quantity of a therapeutic additive including an herbal compound. For example, the administering may include one or more of consuming, applying on skin and spraying on skin. Further, each of the first quantity and the second quantity may correspond to a predetermined level of complexing of cyclodextrin compound with the therapeutic additive. Further, the disease may be one or more of obesity, type II diabetes, and dyslipidaemia.

According to some embodiments, the present disclosure relates to a nutraceutical formulation including a nutrient and a therapeutic mixture including a first quantity of cyclodextrin compound and a second quantity of a therapeutic additive including an herbal compound. Further, each of the first quantity and the second quantity may correspond to a predetermined level of complexing of cyclodextrin compound with the therapeutic additive.

Although the invention has been explained in relation to its preferred embodiment, it is to be understood that many other possible modifications and variations can be made without departing from the spirit and scope of the invention.

I/We claim:

1. A method of preparing a therapeutic mixture comprising an herbal compound, wherein the method comprises:
selecting, using a first selector unit, a cyclodextrin compound;
selecting, using a second selector unit, a therapeutic additive comprising the herbal compound;
determining, using a control unit, each of a first quantity of cyclodextrin compound and a second quantity of the therapeutic additive corresponding to a predetermined level of complexing of cyclodextrin compound; and
complexing, using a mixer unit, the first quantity of cyclodextrin compound and the second quantity of the therapeutic additive to form the therapeutic mixture.

2. The method of claim 1, wherein the herbal compound comprises at least one of Piperine, Reseveratrol, Coenzyme Q10, Niaziridin, Glycyrrhizin, Cuminum Cyminum, Carum Carvi, Alicillin, Alicillin & Cu2+, Lysergol, Aloe Vera Gel, Aloe Vera whole leaf extract, Stevia Rebaudiana, Curcumin, Sinomenine, Genistein, Ammannia Multiflora, Capsaicin, Quercetin and Naringin.

3. The method of claim 1, wherein the cyclodextrin compound comprises at least one of cyclodextrin alpha, cyclodextrin beta, and cyclodextrin gamma.

4. The method of claim 1, wherein the cyclodextrin compound comprises alpha cyclodextrin, wherein the first quantity of cyclodextrin compound is determined to effect at least one of lowering a glycemic index of a food, lowering blood glucose level, lowering of Low-density lipoprotein (LDL), lowering of triglycerides and a weight loss in a user consuming the therapeutic mixture.

5. The method of claim 1, wherein the predetermined level of complexing of cyclodextrin compound comprises a maximum level.

6. The method of claim 1 further comprising complexing, using the mixer unit, a third quantity of cyclodextrin compound with at least one of an omega fatty acid, an essential oil and a fat soluble vitamin.

7. The method of claim 1 further comprising adding, using the mixer unit, at least one of a nutrient and a medication to the therapeutic mixture, wherein the herbal compound enhances bioavailability of at least one of the nutrient and the medication.

8. The method of claim 7 further comprising complexing, using the mixer unit, a fourth quantity of cyclodextrin compound with at least one of the nutrient and the medication.

9. The method of claim 1, wherein at least one of the selecting of the cyclodextrin compound and determining the first quantity of cyclodextrin compound is performed to provide protection of the therapeutic mixture from at least one environmental disturbance.

10. An apparatus for preparing a therapeutic mixture comprising an herbal compound, wherein the apparatus comprises:
a first selector unit configured for selecting a cyclodextrin compound;
a second selector unit configured for selecting a therapeutic additive comprising the herbal compound;
a control unit configured for determining each of a first quantity of cyclodextrin compound and a second quantity of the therapeutic additive corresponding to a predetermined level of complexing of cyclodextrin compound; and
a mixer unit configured for complexing the first quantity of cyclodextrin compound and the second quantity of the therapeutic additive to form the therapeutic mixture.

11. The apparatus of claim 10, wherein the mixer unit is further configured for complexing a third quantity of cyclodextrin compound with at least one of an omega fatty acid, an essential oil and a fat soluble vitamin.

12. A therapeutic mixture comprising an herbal compound, wherein the therapeutic mixture comprises a first quantity of cyclodextrin compound and a second quantity of a therapeutic additive comprising the herbal compound, wherein each of the first quantity and the second quantity correspond to a predetermined level of complexing of cyclodextrin compound with the therapeutic additive.

13. The therapeutic mixture of claim 12 further comprising at least one of a nutrient and a medication, wherein the herbal compound enhances bioavailability of at least one of the nutrient and the medication.

14. The therapeutic mixture of claim 12 further comprising a fourth quantity of cyclodextrin compound complexed with at least one of the nutrient and the medication.

15. The therapeutic mixture of claim 12, wherein the herbal compound comprises at least one of Piperine, Reseveratrol, Coenzyme Q10, Niaziridin, Glycyrrhizin, Cuminum Cyminum, Carum Carvi, Alicillin, Alicillin & Cu2+, Lysergol, Aloe Vera Gel, Aloe Vera whole leaf extract, Stevia Rebaudiana, Curcumin, Sinomenine, Genistein, Ammannia Multiflora, Capsaicin, Quercetin and Naringin.

16. The therapeutic mixture of claim 12, wherein the cyclodextrin compound comprises at least one of cyclodextrin alpha, cyclodextrin beta, and cyclodextrin gamma.

17. The therapeutic mixture of claim 12, wherein the cyclodextrin compound comprises alpha cyclodextrin, wherein the first quantity of cyclodextrin compound is adequate to effect at least one of lowering a glycemic index of a food, lowering blood glucose level, lowering of Low-density lipoprotein (LDL), lowering of triglycerides and a weight loss in a user consuming the therapeutic mixture.

18. The therapeutic mixture of claim 12, wherein the predetermined level of complexing of cyclodextrin compound comprises a maximum level.

19. The therapeutic mixture of claim 12 further comprising a third quantity of cyclodextrin compound complexed with at least one of an omega fatty acid, an essential oil and a fat soluble vitamin.

20. The therapeutic mixture of claim 12 wherein the first quantity of cyclodextrin compound is adequate to provide a predetermined level of protection of the therapeutic mixture from at least one environmental disturbance.
